(19) United States
(12) Patent Application Publication
(10) Pub. No.: US 2024/0206517 A1
(43) Pub. Date: Jun. 27, 2024

(54) METHOD

(71) Applicant: SOCIETE DES PRODUITS NESTLE S.A., Vevey (CH)

(72) Inventors: TSZ NING MAK, Singapore (SG); CAROLINE LE ROY, Lausanne (CH); LISA LAMOTHE, Lausanne 26 (CH); PALOMA ELORTEGUI PASCUAL, Epalinges (CH)

(21) Appl. No.: 18/577,113

(22) PCT Filed: Jul. 8, 2022
(86) PCT No.: PCT/EP2022/069022

§ 371 Date: Jan. 5, 2024

(30) Foreign Application Priority Data
Jul. 9, 2021 (EP) 21184919.5

(51) Int. Cl. A23L 33/21; G01N 33/02

(52) U.S. Cl. CPC A23L 33/21; G01N 33/02

(57) ABSTRACT

The present invention relates to a method for determining the effects of a food, meal, or diet on a subject's gut microbiota, wherein the method comprises determining the sum of the different types of dietary fibre present in the food, meal, or diet.

### FIELD OF THE INVENTION

The present invention relates to a method for determining the effects of a food, meal, or diet on a subject's gut microbiota, comprising determining the sum of the different types of dietary fibre present in the food, meal, or diet. The present invention further relates to a dietary index for assessing the effects of a food, meal, or diet on a subject's gut microbiota.

### BACKGROUND TO THE INVENTION

The diversity and composition of the microbial community residing in the large intestine (gut microbiome) is tightly linked to the health status of its host (Lynch & Pedersen, 2016, New England Journal of Medicine, 375(24), 2369-2379). The composition of this dynamic ecosystem is partly shaped by environmental factors including diet (Zhernakova et al., 2016, Science, 352(6285), 565-569; Falony et al., 2016, Science, 352(6285), 560-564).

Within diet, some components such as fibre are recognised to have effects on the gut microbiome (Morrison et al., 2020, Microbiome, 8(15)), including the infant gut microbiome at weaning (Laursen et al., 2016, Msphere, 1(1)). Dietary fibre is one of the most diverse groups of associated molecules encountered in food: types of fibre can vary according to their sugar and linkage types, chain length, particle size or sources (Hamaker et al., 2014, Journal of molecular biology, 426(23), 3838-3850). This variability in the structure of dietary fibres can selectively impact the gut microbiota.

Studies investigating the effect of fibre consumption on the gut microbiome have so far focused on the implication of total fibre consumption or single fibre polymers without questioning the effects of fibre diversity on the microbial ecosystem. The majority of dietary recommendations for increased fibre intake are based solely on single types of fibre that tend to reduce gut microbiome diversity. Hence, ignoring the variability in fibre structure when studying the gut microbiome prevents full understanding of the role of fibre on gut microbiome composition.

### SUMMARY OF THE INVENTION

The inventors have developed a system which assess the diversity of dietary fibres in the diet/food product to support the development/maintenance of a healthy microbiome.

The inventors have shown that the system can be used to develop a new dietary index capturing the diversity of fibre content of a food, a meal or a diet. This fibre diversity index can be used to evaluate the richness of the fibre source available for fermentation by gut microbes, and which reflects the potential of a product, meal or diet to enhance gut microbiome diversity and the growth of beneficial taxa. The fibre index may be used to facilitate adjustments to diet recommendation to ensure a high diversity of fibre intake through the diet.

In one aspect, the present invention provides a method for determining the effects of a food, meal, or diet on a subject's gut microbiota, wherein the method comprises determining the sum of the different types of dietary fibre present in the food, meal, or diet.

The method may further comprise determining the amount of fibre present in the food, meal or diet. In some embodiments, the method comprises determining the total amount of dietary fibre present in the food, meal, or diet. In some embodiments, the method further comprises determining the amount of each different type of dietary fibre present in the food, meal, or diet.

A greater sum of the different types of dietary fibre present in the food, meal, or diet provides a more diverse gut microbiota.

In some embodiments, a more diverse gut microbiota is a higher alpha diversity, preferably wherein the alpha diversity is determined using a richness index, a phylogenetic diversity index, or a Shannon index.

In some embodiments, the different types of dietary fibre comprise six or more types of soluble or insoluble dietary fibre. In some embodiments, the different types of dietary fibre comprise or consist of: cellulose, resistant starch, mix-linkage glucans, hemicellulose, arabinoxylan, xyloglucan, galactomannans, pectins, non-digestible oligosaccharides, fructooligosaccharide, galactooligosaccharide, and gums.

The method may further comprise determining a dietary index using the sum of the different types of dietary fibre present in the food, meal, or diet. In some embodiments the dietary index comprises or consists of the sum of the different types of dietary fibre present in the food, meal, or diet. In some embodiments, the total amount of dietary fibre present in the food, meal, or diet is also used to determine the dietary index. In some embodiments, the dietary index comprises the total amount of dietary fibre present in the food, meal, or diet.

In some embodiments, the amount of each different type of dietary fibre present in the food, meal, or diet is also used to determine the dietary index. The dietary index may comprise the amount of each different type of dietary fibre present in the food, meal, or diet.

A higher dietary index referred to herein may provide a more diverse gut microbiota, preferably wherein a more diverse gut microbiota has a higher alpha diversity. The alpha diversity is determined using a richness index, a phylogenetic diversity index, or a Shannon index.

In another aspect, the present invention provides a dietary index for assessing the effects of a food, meal, or diet on a subject's gut microbiota. The dietary index may be determined using a method of the invention.

In another aspect, the present invention provides a method for providing a dietary index as disclosed herein, wherein the method comprises determining the dietary index using the sum of the different types of dietary fibre present in a food, meal, or diet.

In another aspect, the present invention provides a method for maintaining or improving a subject's gut microbiota diversity, wherein the method comprises:
(a) determining the effects of a food, meal, or diet on a subject's gut microbiota using a method as disclosed herein; and
(b) adjusting the subject's present diet to provide an adjusted diet with an improved effect on the subject's gut microbiota diversity.

In another aspect, the present invention provides a method for maintaining or improving a subject's gut microbiota diversity, wherein the method comprises:
(a) determining a dietary index as disclosed herein for the subject's present diet; and
(b) adjusting the subject's present diet to provide an adjusted diet with an improved dietary index.

The adjusted diet may provide a greater number of different types of dietary fibre than the subject's non-adjusted diet. After adjusting the diet, the gut microbiota status of the subject may be healthy. The adjusted diet may increase the abundance and/or function of favourable microbial taxa and/or may decrease the abundance and/or function of unfavourable microbial taxa.

In another aspect, the present invention provides use of the sum of the different types of dietary fibre present in a food, meal, or diet to assess the effects of the food, meal, or diet on a subject's gut microbiota.

In another aspect, the present invention provides use of a dietary index as disclosed herein, for assessing the effects of a food, meal, or diet on a subject's gut microbiota.

In another aspect, the present invention provides a computer program comprising instructions which, when the program is executed by a computer, cause the computer to carry out a method as disclosed herein.

In another aspect, the present invention provides a computer program comprising instructions which, when the program is executed by a computer, cause the computer to determine a dietary index for assessing the effects of a food, meal, or diet on a subject's gut microbiota, given the different types of dietary fibre present in the food, meal, or diet.

In another aspect, the present invention provides a computer-readable medium comprising instructions which, when executed by a computer, cause the computer to carry out a method as disclosed herein.

In another aspect, the present invention provides a computer-readable medium comprising instructions which, when executed by a computer, cause the computer to determine a dietary index for assessing the effects of a food, meal, or diet on a subject's gut microbiota, given the different types of dietary fibre present in the food, meal, or diet.

In some embodiments, the subject is an infant or a toddler.

### DETAILED DESCRIPTION OF THE INVENTION

Various preferred features and embodiments of the present invention will now be described by way of non-limiting examples. The skilled person will understand that they can combine all features of the invention disclosed herein without departing from the scope of the invention as disclosed.

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including” or “includes”; or “containing” or “contains”, and are inclusive or open-ended and do not exclude additional, non-recited members, elements or steps. The terms “comprising”, “comprises” and “comprised of” also include the term “consisting of”.

Numeric ranges are inclusive of the numbers defining the range.

The publications discussed herein are provided solely for their disclosure prior to the filing date of the present application. Nothing herein is to be construed as an admission that such publications constitute prior art to the claims appended hereto.

### Fibre

The present invention provides a method for determining the effects of a food, meal, or diet on a subject's gut microbiota, wherein the method comprises determining the sum of the different types of dietary fibre present in the food, meal, or diet.

Dietary fibre can be categorised as “soluble fibre” or “insoluble fibre”. Some types of both soluble and insoluble fibre can be fermented to short chain fatty acids (SCFAs).

Soluble fibre dissolves in water and is generally viscous. Regular intake of soluble fibres can lower blood levels of LDL cholesterol, a risk factor for cardiovascular diseases. Examples of soluble fibre include mucilage, beta glucans, inulin oligofructose, pectins and gums, polydextrose polyols, psyllium, resistant starch and wheat dextrin.

Insoluble fibre does not dissolve in water and adds bulk to fecal material. Examples of insoluble fibre include cellulose, hemicellulose and lignin.

Properties of these types of fibres are well known in the art, for example as described in Dhingra et al., 2012, J Food Sci Technol, 49(3): 255-266.

Examples of types of fibre are well-known in the art, for example as described in Dhingra et al., 2012, J Food Sci Technol, 49(3): 255-266 (e.g. Table 1) and Stephen et al. 2017, Nutrition Research Reviews, 300, 149-190 (e.g. Tables 3 and 6). Further specific examples of types of fibre are provided in Table 1 below.

TABLE 1
Examples of types of dietary fibre

| Category | Type | Examples | Where found |
|---|---|---|---|
| Insoluble | Beta-glucans | Cellulose | Cereals, fruits, vegetables |
| | | Chitin | Fungi, exoskeleton of insects and crustaceans |
| | Hemicelluloses | Hexoses (e.g. mannose, | Cereals, bran, timber, legumes e.g. wheat, barley |
| | | glucose and galactose) | |
| | | Pentose (e.g. xylose | Cereals, bran, timber, legumes e.g. rye, oat |
| | | and arabinose) | |
| | Lignins | E.g. coniferyl alcohol, | Stones of fruits, vegetables, cereals |
| | | sinapyl alcohol, | |
| | | paracoumaryl alcohol | |
| | Resistant | E.g. RS1, SR2, RS3, | High amylose corn and wheat, barley, legumes, |
| | starches | RS4, RS5 | raw bananas, cooked and cooled pasta and |
| | | | potatoes |
| | Gums | Xanthan gum | Xanthomonas bacteria |
| Soluble | Hemicelluloses | Arabinoxylan | Psyllium |
| | | Galactomannan (e.g. | Leguminous seeds, guar beans, locus bean gum, |
| | | guar gum) | fenugreek, alfalfa |
| | | Glucomannan | Plants e.g. konjac, salep, conifers, dicotyledons |
| | | Mix-linkage glucans | Cereals |
| | | Xyloglucan | Plants, e.g. tamarind seed |
| | Fructans | Inulins | Plants e.g. chicory |
| | Gums | Gum arabic | Acacia sap |
| | Non-digestible | Fructooligosaccharides | Plants e.g. onion, chicory garlic, asparagus, |
| | oligosaccharides | | banana, artichoke |
| | | Galactooligosaccharides | Legumes, nuts, soy beans, diary products |
| | Polydextrose | | Synthetic |
| | Polyuronides | Pectins (e.g. | Fruit skin, vegetables |
| | | homogalacturonans, | |
| | | rhamnofalacturonans) | |
| | | Alginates (e.g. sodium, | Algae |
| | | potassium, ammonium, | |
| | | calcium, propylene | |
| | | glycol, agar and | |
| | | carrageen) | |
| | Raffinoses | E.g. trisacharide | Legumes |
| | | raffinose, | |
| | | tetrasaccharide | |
| | | stachyose, | |
| | | pentasaccharide | |
| | | verbascose | |

Many common foods have had their fibre polymers categorised. For example, see Stephen et al. 2017, Nutrition Research Reviews, 300, 149-190 (e.g. Table 4) and Dhingra et al., 2012, J Food Sci Technol, 49(3): 255-266 (e.g. Table 2). See also Table 2 below.

TABLE 2
Fibre polymer categories for food items. Fibre polymer categories were assigned
based on the monosaccharide composition of non-starch polysaccharides in each food item.

| Food item | Fibre polymer category | References |
|---|---|---|
| Cabbage | Pectins | Pectic polysaccharides of cabbage (Brassica oleracea). |
| | | Phytochemistry, Volume 23, Issue 1, 1984, Pages 107-115 |
| | Pectins | The isolation and analysis of cell wall material from the |
| | XyloGlucans | alcohol-insoluble residue of cabbage (Brassica oleracea |
| | Cellulose | var. capitata) |
| Carrot | Pectin | PENNER, M. H., & KIM, S. (1991). Nonstarch |
| | Cellulose | polysaccharide fractions of raw, processed and cooked |
| | | carrots. Journal of food science, 56(6), 1593-1596. |
| | Pectin | Nyman, E. M. G. L., & Svanberg, S. M. (2002). Modification |
| | | of physicochemical properties of dietary fibre in carrots by |
| | | mono-and divalent cations. Food Chemistry, 76(3), 273- |
| | | 280. |
| | Pectin | Bao, B., & Chang, K. C. (1994). Carrot pulp chemical |
| | Cellulose | composition, color, and water-holding capacity as affected |
| | | by blanching. Journal of Food Science, 59(6), 1159-1161. |
| Pea | XyloGlucans | Hayashi, T., Marsden, M. P., & Delmer, D. P. (1987). Pea |
| | | xyloglucan and cellulose: VI. Xyloglucan-cellulose |
| | | interactions in vitro and in vivo. Plant Physiology, 83(2), |
| | | 384-389. |
| | Cellulose | Hayashi, T., Marsden, M. P., & Delmer, D. P. (1987). Pea |
| | | xyloglucan and cellulose: VI. Xyloglucan-cellulose |
| | | interactions in vitro and in vivo. Plant Physiology, 83(2), |
| | | 384-389. |
| | Raffinose | Peterbauer, T., Lahuta, L. B., Blöchl, A., Mucha, J., Jones, |
| | | D. A., Hedley, C. L., . . . & Richter, A. (2001). Analysis of the |
| | | raffinose family oligosaccharide pathway in pea seeds with |
| | | contrasting carbohydrate composition. Plant Physiology, |
| | | 127(4), 1764-1772. |
| | Oligosaccharides | Jones, D. A., DuPont, M. S., Ambrose, M. J., Frías, J., & |
| | | Hedley, C. L. (1999). The discovery of compositional |
| | | variation for the raffinose family of oligosaccharides in pea |
| | | seeds. Seed Science Research, 9(4), 305-310. |
| Spinach | Cellulose | Macarisin, D., Patel, J., Bauchan, G., Giron, J. A., & |
| | Pectin | Sharma, V. K. (2012). Role of curli and cellulose expression |
| | | in adherence of Escherichia coli O157: H7 to spinach |
| | | leaves. Foodborne pathogens and disease, 9(2), 160-167. |
| Beetroot | Cellulose | Wen, L. F., Chang, K. C., Brown, G., & Gallaher, D. D. |
| | XyloGlycans | (1988). Isolation and characterization of hemicellulose and |
| | Pectin | cellulose from sugar beet pulp. Journal of Food Science, |
| | | 53(3), 826-829. |
| | Cellulose | Mikihiko Bayashi, Kazumi Funane, Hiromasa Ueyama, |
| | XyloGlycans | Setsuko Ohya, Masakatsu Tanaka & Yoji Kato (1993) |
| | Pectin | Sugar Composition of Beet Pulp Polysaccharides and Their |
| | | Enzymatic Hydrolysis, Bioscience, Biotechnology, and |
| | | Biochemistry, 57:6, 998-1000, DOI: 10.1271/bbb.57.998 |
| Sweet potato | Cellulose | Mei, X., Mu, T.H. & Han, J.J. (2010). Composition and |
| | Pectin | physicochemical properties of dietary fiber extracted from |
| | Xyloglucans | residues of 10 varieties of sweet potato by a sieving |
| | | method. Journal of Agricultural and Food Chemistry, 58, |
| | | 7305-7310. |
| | | Takamine, K., Abe, J. I., Iwaya, A., Shimono, K., Maseda, |
| | | S., & Hizukuri, S. (2000). Preparation of pectin from sweet |
| | | potato residue and its characterization. Journal of Applied |
| | | Glycoscience, 47(2), 201-206. |
| Turnip | Cellulose | Obregón-Cano, S., Moreno-Rojas, R., Jurado-Millán, A. M., |
| | XyloGlucans | Cartea-González, M. E., & Haro-Bailón, D. (2019). Analysis |
| | Pectin | of the acid detergent fibre content in turnip greens and |
| | | turnip tops (Brassica rapa L. Subsp. rapa) by means of |
| | | near-infrared reflectance. Foods, 8(9), 364. |
| Apple | Pectins (arabinans, RG-I) | Aspinall, G. O., & Fanous, H. K. (1984). Structural |
| | | investigations on the non-starchy polysaccharides of |
| | | apples. Carbohydrate polymers, 4(3), 193-214. |
| | XyloGlucans (fucogalacto-) | Voragen, F. G., Schols, H. A., & Pilnik, W. (1986). Structural |
| | | features of the hemicellulose polymers of apples. Zeitschrift |
| | | für Lebensmittel-Untersuchung und Forschung, 183(2), |
| | | 105-110. |
| | Pectins (arabinans) | Mehrländer, K., Dietrich, H., Sembries, S., Dongowski, G., |
| | XyloGlucans | & Will, F. (2002). Structural characterization of |
| | Starch | oligosaccharides and polysaccharides from apple juices |
| | | produced by enzymatic pomace liquefaction. Journal of |
| | | agricultural and food chemistry, 50(5), 1230-1236. |
| Pear | Pectins | Raffo, M. D., Ponce, N. M., Sozzi, G. O., Vicente, A. R., & |
| | XyloGlucans | Stortz, C. A. (2011). Compositional changes in ‘Bartlett’pear |
| | Starch | (Pyrus communis L.) cell wall polysaccharides as affected |
| | | by sunlight conditions. Journal of agricultural and food |
| | | chemistry, 59(22), 12155-12162. |
| | Pectins (arabinans) | Raffo, M. D., Ponce, N. M., Sozzi, G. O., Stortz, C. A., & |
| | Starch | Vicente, A. R. (2012). Changes on the cell wall composition |
| | | of tree-ripened “Bartlett” pears (Pyrus communis L.). |
| | | Postharvest biology and technology, 73, 72-79. |
| Plum | Pectins | Kosmala, M., Milala, J., Kołodziejczyk, K., Markowski, J., |
| | Cellulose | Zbrzeźniak, M., & Renard, C. M. (2013). Dietary fiber and |
| | | cell wall polysaccharides from plum (Prunus domestica L.) |
| | | fruit, juice and pomace: Comparison of composition and |
| | | functional properties for three plum varieties. Food research |
| | | international, 54(2), 1787-1794. |
| | Pectins | Renard, C. M., & Ginies, C. (2009). Comparison of the cell |
| | Cellulose | wall composition for flesh and skin from five different plums. |
| | XyloGlucans (fucogalacto-) | Food Chemistry, 114(3), 1042-1049. |
| Banana | Cellulose | Cordenunsi, B. R., Shiga, T. M., & Lajolo, F. (2008). Non- |
| | GlucuronoArabinoxylan | starch polysaccharide composition of two cultivars of |
| | Pectin (galacturonan) | banana (Musa acuminata L.: cvs Mysore and Nanicão). |
| | Glucomannan | Carbohydrate polymers, 71(1), 26-31. |
| | | Shiga, T. M., Soares, C. A., Nascimento, J. R., Purgatto, E., |
| | | Lajolo, F. M., & Cordenunsi, B. R. (2011). Ripening- |
| | | associated changes in the amounts of starch and non- |
| | | starch polysaccharides and their contributions to fruit |
| | | softening in three banana cultivars. Journal of the Science |
| | | of Food and Agriculture, 91(8), 1511-1516. |
| Potato | Pectin (RG-I) | Ring, S. G., & Selvendran, R. R. (1978). Purification and |
| | | methylation analysis of cell wall material from Solanum |
| | | tuberosum. Phytochemistry, 17(4), 745-752. |
| | XyloGlucan (arabinogalacto-) | Ring, S. G., & Selvendran, R. R. (1981). An |
| | | arabinogalactoxyloglucan from the cell wall of Solanum |
| | | tuberosum. Phytochemistry, 20(11), 2511-2519. |
| | Pectin | van Marle, J. T., Recourt, K., van Dijk, C., Schols, H. A., & |
| | | Voragen, A. G. (1997). Structural features of cell walls from |
| | | potato (Solanum tuberosum L.) cultivars Irene and Nicola. |
| | | Journal of agricultural and food chemistry, 45(5), 1686- |
| | | 1693. |
| | Cellulose | Structural features of cell-wall polysaccharides of potato |
| | Pectin | (Solanum tuberosum). Carbohydrate Research, ISSN: |
| | XyloGlucan | 0008-6215, Vol: 195, Issue: 2, Page: 257-272 |
| | | Publication Year: 1990 |

Routine methods for determining fibre content of food are known in the art, for example as described in Mertens, 2003, J. Anim. Sci. 81:3233-3249, Dhingra et al., 2012, J Food Sci Technol, 49(3): 255-266 and Stephen et al., 2017, Nutrition Research Reviews, 300, 149-190. Specific examples include the modified Englyst method, which involves enzymatic-chemical extraction and fractionation of the non-starch polysaccharide (NSP) and their subsequent determination as neutral sugars by gas-liquid chromatography (GLC) (Englyst et al., 1982, Analyst, 107:307-318).

The different types of dietary fibre may be chemically distinct from each other. In some embodiments of the invention, the different types of dietary fibre comprise two or more, three or more, five or more, six or more, seven or more, eight or more, nine or more, or ten or more types of soluble or insoluble dietary fibre. Preferably, the different types of dietary fibre comprise six or more types of soluble or insoluble dietary fibre.

In some embodiments of the present invention, the different types of dietary fibre comprise or consist of: cellulose, resistant starch, mix-linkage glucans, hemicellulose, arabinoxylan, xyloglucan, galactomannans, pectins, non-digestible oligosaccharides, fructooligosaccharide, galactooligosaccharide, and gums.

### Microbiota and Microbiome

A greater sum of the different types of dietary fibre present in the food, meal, or diet may provide a more diverse gut microbiota.

Additionally, a greater total amount of dietary fibre present in the food, meal, or diet and/or a greater amount of each different type of dietary fibre present in the food, meal, or diet may provide a more diverse gut microbiota.

The “gut microbiota” is the composition of microorganisms (including bacteria, archaea and fungi) that live in the digestive tract.

The term “gut microbiome” may encompass both the “gut microbiota” and their “theatre of activity”, which may include their structural elements (nucleic acids, proteins, lipids, polysaccharides), metabolites (signalling molecules, toxins, organic, and inorganic molecules), and molecules produced by coexisting hosts and structured by the surrounding environmental conditions (see e.g. Berg, G., et al., 2020. Microbiome, 8(1), pp. 1-22).

In the present invention, the term “gut microbiome” may therefore be used interchangeably with the term “gut microbiota”.

### Gut Microbiota Diversity

A subject's “gut microbiota diversity” may refer to the number of different taxa present in the gut microbiome and/or stool of the subject (e.g. “richness”). It may also refer to the “evenness” of the gut microbiome and/or stool of the subject, i.e. takes into account the abundance or relative abundance of each taxon.

Alpha diversity may be the diversity of a single sample (such as a fecal sample), and can take into account the number of different taxa and their relative abundances. Alpha diversity can be determined using a richness index, a phylogenetic diversity index, or a Shannon index. These indexes can be determined using methods routine in the art, such as, for example, using the R package Phyloseq (McMurdie and Holmes, 2013, PLoS One, 8, Article e61217). Alpha diversity indexes may be calculated based on 16 rRNA sequencing data and/or whole genome shotgun metagenomics sequencing.

Beta diversity can be determined using a Whittaker index (e.g. Jaccard or Sorensen), a Min-Max Index (e.g. Simpson, β-2 or β-3), a Cody Index or an Abundance index (e.g. Bray-Curtis or BD_{TOTAL}). These indexes can be determined using methods routine in the art, such as, for example, using the R package Phyloseq (McMurdie and Holmes, 2013, PLoS One, 8, Article e61217). Beta diversity indexes may be calculated based on 16 rRNA sequencing data and/or whole genome shotgun metagenomics sequencing.

In some embodiments of the invention, a more diverse gut microbiota has a higher alpha diversity, preferably wherein the alpha diversity is determined using a richness index, a phylogenetic diversity index, or a Shannon index.

In some embodiments of the invention, a higher dietary index provides a more diverse gut microbiota, preferably wherein a more diverse gut microbiota has a higher alpha diversity, more preferably wherein the alpha diversity is determined using a richness index, a phylogenetic diversity index, or a Shannon index.

### Gut Microbiota Data

The gut microbiota data of the subject may be determined by any suitable sampling method. For example, gut microbiota data may be determined by any method described in Tang, Q., et al., 2020, Frontiers in cellular and infection microbiology, 10, p. 151.

The gut microbiota data may be determined from fecal samples, endoscopy samples (e.g. biopsy samples, luminal brush samples, laser capture microdissection samples), aspirated intestinal fluid samples, surgery samples, or by in vivo models or intelligent capsule (see e.g. Tang, Q., et al., 2020, Frontiers in cellular and infection microbiology, 10, p. 151).

Suitably, the gut microbiota data may be determined from fecal samples. Fecal samples are naturally collected, non-invasive and can be sampled repeatedly. Fecal materials instantly frozen at −80° C. that can maintain microbial integrity without preservatives have been widely regarded as the gold standard for gut microbiota profiling, but other storage methods with or without preservatives can also be utilised to achieve microbiota compositions similar to those of fresh samples.

The gut microbiota data may be determined from the samples by any suitable detection method. For example, the gut microbiota data may be obtained by or obtainable by sequencing methods (e.g. next-generation sequencing (NGS) methods), PCR-based methods, semi-quantitative detection methods (e.g. from SwissDeCode), cycling temperature capillary electrophoresis (e.g. from REM analytics), cell-based methods, immunological-based methods, or any combination thereof. Preferably, the gut microbiota data is obtained by or obtainable by PCR-based methods, semi-quantitative detection methods (e.g. from SwissDeCode), cycling temperature capillary electrophoresis (e.g. from REM analytics), or immunological-based methods, or any combination thereof.

In some embodiments, the gut microbiota data is determined by sequencing methods (e.g. next-generation sequencing (NGS) methods). NGS enables the profiling of the genomic DNA of all the microorganisms present in a sample. NGS methods can include targeted (e.g. 16S ribosomal RNA sequencing) and/or shotgun sequencing approaches, e.g. as described in Poussin, C., et al., 2018. Drug discovery today, 23(9), pp. 1644-1657.

In some embodiments, the gut microbiota data is determined by PCR-based methods. For example, the gut microbiota data may be obtained by or obtainable by PCR, multiplex PCR (mPCR), and/or quantitative PCR (qPCR). Suitably, the gut microbiota data may be obtained by or obtainable by qPCR, e.g. as described in Jian, C., et al., 2020. PLoS One, 15(1), p.e0227285.

In some embodiments, the gut microbiota data is determined by semi-quantitative detection methods. For example, the gut microbiota data may be determined by culture method, denaturing gradient gel electrophoresis (DGGE), terminal restriction fragment length polymorphism (T-RFLP), fluorescence in situ hybridization (FISH), and/or DNA microarrays, e.g. as described in Fraher, M. H., et al., 2012. Nature reviews Gastroenterology & hepatology, 9(6), p. 312.

In some embodiments, the gut microbiota data is determined by cycling temperature capillary electrophoresis, e.g. as described in Refinetti, P., et al., 2016. Mitochondrion, 29, pp. 65-74.

In some embodiments, the gut microbiota data may be determined by immunological-based methods. Immunological-based methods may be based on antibody-antigen interactions, whereby a particular antibody will bind to its specific antigen and can use polyclonal or monoclonal antibodies. Enzyme-linked immunosorbent assay (ELISA) and lateral flow immunoassay are among the immunological-based methods which can be used, e.g. as described in Law, J. W. F., et al., 2015. Frontiers in microbiology, 5, p. 770. Exemplary methods are described in Amrouche, T., et al., 2006. Journal of microbiological methods, 65(1), pp. 159-170; and Qian, H., et al., 2008. Applied and environmental microbiology, 74(3), pp. 833-839.

In some embodiments, the gut microbiota data is determined by cell-based methods. For example, the gut microbiota data may be determined by counting microbial cells using flow cytometry, e.g. as described in Galazzo, G., et al., 2020. Frontiers in cellular and infection microbiology, 10, p. 403.

In some embodiments, the gut microbiota data is determined by a combination of one or more methods described herein, e.g. as described in Allaband, C., et al., 2019. Clinical Gastroenterology and Hepatology, 17(2), pp. 218-230.

The gut microbiota data may provide the relative abundance and/or absolute abundance for the plurality of microbial taxa. Suitably, the gut microbiota data provides the relative abundance for the plurality of microbial taxa.

The microbial taxa may be classified according to a suitable classification, see e.g. Pitt, T. L. and Barer, M. R., 2012. Medical Microbiology, p. 24. The microbial taxa may be taxonomically-classified and/or functionally-classified.

In some embodiments, the microbial taxa are taxonomically-classified. Microbial taxonomy refers to the rank-based classification of microbes. In the scientific classification established by Carl Linnaeus, each species has to be assigned to a genus, which in turn is a lower level of a hierarchy of ranks (family, suborder, order, subclass, class, division/phyla, kingdom and domain). Prokaryotic taxa which have been correctly described are reviewed in e.g. Bergey's manual of Systematic Bacteriology.

Suitably, the microbial taxa in the microbial ratios are taxonomically-classified by phylum, class, order, family, genus, species and/or strains. Suitably, the microbial taxa in the microbial ratios are taxonomically-classified by phylum, genus and/or species. Suitably, the microbial taxa in the microbial ratios are taxonomically-classified by genus and/or species. In some embodiments, the microbial taxa in the microbial ratios are taxonomically-classified by genus. In some embodiments, the microbial taxa in the microbial ratios are taxonomically-classified by species.

In some embodiments, the microbial taxa are functionally-classified. For example, the microbial taxa may be classified by one or more phenotypic classification systems (e.g. gram stain, morphology, growth requirements, biochemical reactions, serologic systems, environmental reservoirs etc.). In some embodiments, the microbial taxa are classified according to biological or metabolic pathways, protein domains or families, functional modules, complex carbohydrate metabolism, antibiotic resistance, virulence factors, bacterial drug targets and endotoxins, mobile genetic elements, and/or any other functional properties, such as those described in Kultima, J. R., et al., 2016. Bioinformatics, 32(16), pp. 2520-2523 and Overbeek, R., et al., 2014. Nucleic acids research, 42(D1), pp. D206-D214.

Suitably, the microbial taxa are bacterial taxa.

### Dietary Index

A dietary index provides a summary measure of a characteristic. A fibre dietary index provides a summary measure of fibre within a food, meal or diet. A fibre dietary index according to the present invention is based, at least in part, on the sum of different types of fibre present in the food, meal or diet.

Thus, the present invention further comprises determining a dietary index using the sum of the different types of dietary fibre present in the food, meal, or diet.

In some embodiments of the invention, the dietary index comprises or consists of the sum of the different types of dietary fibre present in the food, meal, or diet.

In some embodiments of the invention, the total amount of dietary fibre present in the food, meal, or diet is also used to determine the dietary index.

In some embodiments of the invention, the amount of each different type of dietary fibre present in the food, meal, or diet is also used to determine the dietary index.

### Methods for Maintaining or Improving Gut Microbiota Diversity

The present invention provides a method for maintaining or improving a subject's gut microbiota diversity, wherein the method comprises:
(a) determining the effects of a food, meal, or diet on a subject's gut microbiota using a method as disclosed herein; and
(b) adjusting the subject's present diet to provide an adjusted diet with an improved effect on the subject's gut microbiota diversity.

In another aspect, the present invention provides a method for maintaining or improving a subject's gut microbiota diversity, wherein the method comprises:
(a) determining a dietary index as disclosed herein for the subject's present diet; and
(b) adjusting the subject's present diet to provide an adjusted diet with an improved dietary index.

### Maintaining or Improving Out Microbiota Diversity

Maintaining gut microbiota diversity refers to the gut microbiota diversity not being significantly reduced. Improving gut microbiota diversity may refer to increasing the gut microbiota diversity.

An improved gut microbiota diversity may refer to a more “rich” and/or “even” gut microbiota, for example as determined by an Alpha diversity index such as a richness index, a phylogenetic diversity index, or a Shannon index.

Suitably, after adjusting the diet of the subject, the subject may have a more diverse gut microbiota.

Improving the gut microbiota diversity may improve the functioning of the gut microbiome.

In some embodiments, after adjusting the diet, the subject may be in an appropriate gut maturation state, in an appropriate gut progression state, and/or in an appropriate gut succession stage. An “appropriate gut maturation state” may mean that the subject's gut microbiota is maturing normally or properly. An “appropriate gut progression state” may mean that the subject's gut microbiota is progressing or evolving in a timely manner. An “appropriate gut succession state” may mean that the subject's gut microbiota is succeeding in a timely manner.

In some embodiments, after adjusting the diet, the subject may be at decreased risk of suffering a disease, disorder or condition associated with the gut microbiome, such as such as Irritable Bowel Syndrome, Inflammatory Bowel Disease, allergy, diabetes, cancer, asthma, and obesity. In some embodiments, after adjusting the diet, the subject may be at decreased risk of suffering a disease, disorder or condition associated with fibre intake, such as cardiovascular disease, constipation, diverticular disease, oesophageal cancer, gastric cancer, colorectal adenomas and colorectal cancer, breast cancer, endometrial cancer, prostate cancer, pancreatic cancer, ovarian cancer, renal cancer.

In some embodiments, after adjusting the diet, the subject may experience reduced symptoms of a disease, disorder or condition associated with the gut microbiome, such as such as Irritable Bowel Syndrome, Inflammatory Bowel Disease, allergy, diabetes, cancer, asthma, and obesity. In some embodiments, after adjusting the diet, the subject may experience reduced symptoms of a disease, disorder or condition associated with fibre intake, such as cardiovascular disease, constipation, diverticular disease, oesophageal cancer, gastric cancer, colorectal adenomas and colorectal cancer, breast cancer, endometrial cancer, prostate cancer, pancreatic cancer, ovarian cancer, renal cancer.

### Food, Meal or Diet

A “food” may include a single food item consumed by the subject. A “meal” may include all the food items consumed in a single meal by the subject. The subject's “diet” may include all the food consumed by the subject.

The subject's diet may provide a plurality of food groups. The term “food group” refers to a collection of foods that share similar nutritional properties or biological classifications. Nutrition guides typically divide foods into food groups and recommend daily servings of each group for a healthy diet. Exemplary food groups include fruits; vegetables; pulses, nuts or seeds; meats; starches or grains; dairy; and oils and fats.

The subject's diet may also provide a plurality of food types. The term “food type” may refer to a collection of foods from the same food group that share more similar nutritional properties or biological classifications. Each food group may be further grouped into a plurality of food types. Exemplary food types for the food group fruit can include apples, banana, citrus, berries, other fruits (e.g. pear, peach, pineapple), and dried fruits. Suitable food groups and food types can be readily determined by any suitable method known in the art. For example, suitable food groups and food types can be based on published observations (e.g. Dwyer JT. The Journal of Nutrition. 2018; 148(suppl 3):1575S-80S).

### Computer Programs and Computer-Readable Mediums

The present invention provides a computer program comprising instructions which, when the program is executed by a computer, cause the computer to carry out a method as disclosed herein.

The present invention also provides a computer program comprising instructions which, when the program is executed by a computer, cause the computer to determine a dietary index for assessing the effects of a food, meal, or diet on a subject's gut microbiota, given the different types of dietary fibre present in the food, meal, or diet.

The present invention also provides a computer-readable medium comprising instructions which, when executed by a computer, cause the computer to carry out a method as disclosed herein.

The present invention also provides a computer-readable medium comprising instructions which, when executed by a computer, cause the computer to determine a dietary index for assessing the effects of a food, meal, or diet on a subject's gut microbiota, given the different types of dietary fibre present in the food, meal, or diet.

### Subject

Suitably, the subject is an infant, toddler, or child, preferably wherein the subject is an infant or a toddler.

The term “infant” may refer to a subject aged from 0 years to 1 year, or from 0 months to less than 1 year. The term “toddler” may refer to a subject aged from 1 year to 3 years, or from 1 year to less than 3 years. The term “child” may refer to a subject aged under 18 years. The subject may be an infant, toddler and/or young child. The term “young child” may refer to a subject aged from 3 years to 5 years, or from 3 years to less than 5 years.

Suitably, the subject is 5 years of age or less, 4 years of age or less, 3 years of age or less, 2 years of age or less, 1 year of age or less, or 0.5 years of age or less.

The skilled person will understand that they can combine all features of the invention disclosed herein without departing from the scope of the invention as disclosed.

Preferred features and embodiments of the invention will now be described by way of non-limiting examples.

The practice of the present invention will employ, unless otherwise indicated, conventional techniques of chemistry, biochemistry, molecular biology, microbiology and immunology, which are within the capabilities of a person of ordinary skill in the art. Such techniques are explained in the literature. See, for example, Sambrook, J., Fritsch, E. F. and Maniatis, T. (1989) Molecular Cloning: A Laboratory Manual, 2nd Edition, Cold Spring Harbor Laboratory Press; Ausubel, F. M. et al. (1995 and periodic supplements) Current Protocols in Molecular Biology, Ch. 9, 13 and 16, John Wiley & Sons; Roe, B., Crabtree, J. and Kahn, A. (1996) DNA Isolation and Sequencing: Essential Techniques, John Wiley & Sons; Polak, J. M. and McGee, J.O'D. (1990) In Situ Hybridization: Principles and Practice, Oxford University Press; Gait, M. J. (1984) Oligonucleotide Synthesis: A Practical Approach, IRL Press; and Lilley, D. M. and Dahlberg, J. E. (1992) Methods in Enzymology: DNA Structures Part A: Synthesis and Physical Analysis of DNA, Academic Press. Each of these general texts is herein incorporated by reference.

### EXAMPLES

### Example 1 Fibre Diversity Index

### Material and Methods

### The Cohort

The children involved in this study come from an American cohort called BCP-Enriched. The UNC/UMN Baby Connectome Project (BCP) is an ongoing study jointly conducted by investigators at the University of North Carolina at Chapel Hill and the University of Minnesota and involving a total sample of 500 typically developing infants, toddlers, and preschool-aged children recruited and enrolled between birth and 5 years of age.

BCP-Enriched includes the same 500 children as BCP for whom 24 h dietary recall and fecal samples were collected at several time points in order to assess, among other things, potential interplays among nutrient intakes and gut microbiota in critical period of early development (0 to 3 years of age).

### Intakes and Fibre Index Calculation

The patterns of usual dietary intake and specific information regarding, food, portion, and changes in diet were collected from children between the ages of 0-36 months. At each time point, children completed two days of food records (one weekday and one weekend, if possible). To be considered one of the food records must have been collected 24 hours prior the fecal sample collection date.

Accordingly, 192 24 hours dietary recalls linked to a fecal sample were used in the following analysis. Those 192 observations stand for 105 individual children (mean observation=1.819; SD=1). For each observation, the type of food consumed during the day as well as the cooking methods used were given. Using this information, the fibre diversity index was calculated as the sum of the different types of fibre polymers consumed by each infant during 24 h period preceding sample collection. Alpha diversity metrics and gut microbiome profiling were calculated based on 16 rRNA sequencing data.

Using a linear mixed effect model (“lme4” package in R), the association between the fibre diversity index (positioned as predictor) and gut microbiome diversity (the response: PD whole tree, Shannon and Richness as the observed number of OUT's species) was tested, while a counting for age as fixed effect and individual as a random effect.

### Results

192 observations from 105 individual subjects aged from 0 to 34 months (mean=11.28, SD=6.99) were analysed. Table 3 summarises the distribution of the different variables (Age, Fibre index and three alpha diversity indexes) for the whole population.

TABLE 3
Descriptive table of the variables of interest by age class.

| | TOTAL |
|---|---|
| | (N = 192) |
| Age | |
| Mean (SD) | 11.28 (6.99) |
| Range | 0-34 |
| Fibre index | |
| Mean (SD) | 2.5 (2.45) |
| Range | 0-8 |
| Faith PD | |
| Mean (SD) | 4.24 (0.97) |
| Range | 1.8-6.7 |
| Shannon | |
| Mean (SD) | 5.62 (0.64) |
| Range | 3.2-6.9 |
| Richness | |
| Mean (SD) | 69.9 (29) |
| Range | 16-160 |

We next tested the association between gut microbiome and fibre diversity. The most significant result was observed with richness (beta=2.48, SE=0.85 and P=0.003; Table 4) followed by Shannon (beta=0.05, SE=0.02 and P=0.007) and PD whole tree (beta=0.052, SE=0.025 and P=0.0285.

TABLE 4
Association between the fibre diversity index
and microbiome diversity indexes.

| | BETA | SE | P-VALUE |
|---|---|---|---|
| Pd whole tree | 0.052 | 0.025 | 0.0285 |
| Shannon | 0.050 | 0.018 | 0.0072 |
| Richness | 2.483 | 0.847 | 0.0033 |

### CONCLUSION

We observed that the fibre diversity index was significantly associated with gut microbiome alpha diversity in infants between 0 and 36 months after adjustment for age. These results suggest that the fibre diversity index could be used to evaluate the effects of a food, meal, or diet on the gut microbiome in infants.

### Embodiments

Various preferred features and embodiments of the present invention will now be described with reference to the following numbered paragraphs (paras).
1. A method for determining the effects of a food, meal, or diet on a subject's gut microbiota, wherein the method comprises determining the sum of the different types of dietary fibre present in the food, meal, or diet.
2. The method according to numbered paragraph 1, wherein the method further comprises determining the different types of dietary fibre present in the food, meal, or diet.
3. The method according to numbered paragraph 1 or 2, wherein the method further comprises determining the total amount of dietary fibre present in the food, meal, or diet.
4. The method according to any preceding numbered paragraph, wherein the method further comprises determining the amount of each different type of dietary fibre present in the food, meal, or diet.
5. The method according to any preceding numbered paragraph, wherein a greater sum of the different types of dietary fibre present in the food, meal, or diet provides a more diverse gut microbiota.
6. The method according to any preceding numbered paragraph, wherein a greater total amount of dietary fibre present in the food, meal, or diet and/or a greater amount of each different type of dietary fibre present in the food, meal, or diet provides a more diverse gut microbiota.
7. The method according to numbered paragraph 5 or 6, wherein a more diverse gut microbiota has a higher alpha diversity, preferably wherein the alpha diversity is determined using a richness index, a phylogenetic diversity index, or a Shannon index.
8. The method according to any preceding numbered paragraph, wherein the different types of dietary fibre comprise six or more types of soluble or insoluble dietary fibre.
9 The method according to any preceding numbered paragraph, wherein the different types of dietary fibre comprise or consist of: cellulose, resistant starch, mix-linkage glucans, hemicellulose, arabinoxylan, xyloglucan, galactomannans, pectins, non-digestible oligosaccharides, fructooligosaccharide, galactooligosaccharide, and gums.
10. The method according to any preceding numbered paragraph, wherein the method comprises determining a dietary index using the sum of the different types of dietary fibre present in the food, meal, or diet.
11. The method according to numbered paragraph 10, wherein the dietary index comprises or consists of the sum of the different types of dietary fibre present in the food, meal, or diet.
12. The method according to numbered paragraph 10 or 11, wherein the total amount of dietary fibre present in the food, meal, or diet is also used to determine the dietary index.
13. The method according to any of numbered paragraphs 10-12, wherein the dietary index comprises the total amount of dietary fibre present in the food, meal, or diet.
14. The method according to any of numbered paragraphs 10-13, wherein the amount of each different type of dietary fibre present in the food, meal, or diet is also used to determine the dietary index.
15. The method according to any of numbered paragraphs 10-14, wherein the dietary index comprises the amount of each different type of dietary fibre present in the food, meal, or diet.
16. The method according to any of numbered paragraphs 10-15, wherein a higher dietary index provides a more diverse gut microbiota, preferably wherein a more diverse gut microbiota has a higher alpha diversity, more preferably wherein the alpha diversity is determined using a richness index, a phylogenetic diversity index, or a Shannon index.
17. A dietary index for assessing the effects of a food, meal, or diet on a subject's gut microbiota, wherein the dietary index is determined using the sum of the different types of dietary fibre present in the food, meal, or diet.
18. The dietary index according to numbered paragraph 17, wherein the dietary index comprises or consists of the sum of the different types of dietary fibre present in the food, meal, or diet.
19. The dietary index according to numbered paragraphs 17 or 18, wherein the total amount of dietary fibre present in the food, meal, or diet is also used to determine the dietary index.
20. The dietary index according to any of numbered paragraphs 17-19, wherein the dietary index comprises the total amount of dietary fibre present in the food, meal, or diet.
21. The dietary index according to any of numbered paragraphs 17-20, wherein the amount of each different type of dietary fibre present in the food, meal, or diet is also used to determine the dietary index.
22. The dietary index according to any of numbered paragraphs 17-21, wherein the dietary index comprises the amount of each different type of dietary fibre present in the food, meal, or diet.
23. The dietary index according to any of numbered paragraphs 17-22, wherein the different types of dietary fibre comprise or consist of cellulose, resistant starch, mix-linkage glucans, hemicellulose, arabinoxylan, xyloglucan, galactomannans, pectins, non-digestible oligosaccharides, fructooligosaccharide, galactooligosaccharide, and gums.
24. A method for providing a dietary index according to any of numbered paragraphs 17-23, wherein the method comprises determining the dietary index using the sum of the different types of dietary fibre present in a food, meal, or diet.
25. A method for maintaining or improving a subject's gut microbiota diversity, wherein the method comprises:
   (a) determining the effects of a food, meal, or diet on a subject's gut microbiota using a method according to any of numbered paragraphs 1-16; and
   (b) adjusting the subject's present diet to provide an adjusted diet with an improved effect on the subject's gut microbiota diversity.
26. A method for maintaining or improving a subject's gut microbiota diversity, wherein the method comprises:
   (a) determining a dietary index according to any of numbered paragraphs 17-23 for the subject's present diet; and
   (b) adjusting the subject's present diet to provide an adjusted diet with an improved dietary index.
27. The method according to numbered paragraph 25 or 26, wherein the adjusted diet provides a greater number of different types of dietary fibre than the subject's present diet.
28. Use of the sum of the different types of dietary fibre present in a food, meal, or diet to assess the effects of the food, meal, or diet on a subject's gut microbiota.
29. Use of a dietary index according to any of numbered paragraphs 17-23 for assessing the effects of a food, meal, or diet on a subject's gut microbiota.
30. A computer program comprising instructions which, when the program is executed by a computer, cause the computer to carry out the method according to any of numbered paragraphs 1-16 or 24-27.
31. A computer program comprising instructions which, when the program is executed by a computer, cause the computer to determine a dietary index for assessing the effects of a food, meal, or diet on a subject's gut microbiota, given the different types of dietary fibre present in the food, meal, or diet.
32. A computer-readable medium comprising instructions which, when executed by a computer, cause the computer to carry out the method according to any of numbered paragraphs 1-16 or 24-27.
33. A computer-readable medium comprising instructions which, when executed by a computer, cause the computer to determine a dietary index for assessing the effects of a food, meal, or diet on a subject's gut microbiota, given the different types of dietary fibre present in the food, meal, or diet.
34. The method according to any of numbered paragraphs 1-16 or 24-27, the dietary index according to any of numbered paragraphs 17-23, use according to numbered paragraphs 28 or 29, the computer program according to numbered paragraphs 30 or 31, or the computer-readable medium according to numbered paragraphs 32 or 33, wherein the subject is an infant, toddler, or child, preferably wherein the subject is an infant or a toddler.
35. The method according to any of numbered paragraphs 1-16 or 24-27, the dietary index according to any of numbered paragraphs 17-23, use according to numbered paragraphs 28 or 29, the computer program according to numbered paragraphs 30 or 31, or the computer-readable medium according to numbered paragraphs 32 or 33, wherein the subject is aged about 5 years of age or less, or about 4 years of age or less, or about 3 years of age or less.

All publications mentioned in the above specification are herein incorporated by reference. Various modifications and variations of the disclosed methods, uses, computer-implemented methods and indices of the invention will be apparent to the skilled person without departing from the scope and spirit of the invention. Although the invention has been disclosed in connection with specific preferred embodiments, it should be understood that the invention as claimed should not be unduly limited to such specific embodiments. Indeed, various modifications of the disclosed modes for carrying out the invention, which are obvious to the skilled person are intended to be within the scope of the following claims.

1. A method for determining the effects of a food, meal, or diet on a subject's gut microbiota, wherein the method comprises determining the sum of the different types of dietary fiber present in the food, meal, or diet.

2. The method according to claim 1, wherein a greater sum of the different types of dietary fiber present in the food, meal, or diet provides a more diverse gut microbiota.

3. The method according to claim 1, wherein a more diverse gut microbiota has a higher alpha diversity.

4. The method according to claim 1, wherein the different types of dietary fiber comprise six or more types of dietary fiber.

5. The method according to claim 1, wherein the different types of dietary fiber is selected from the group consisting of: cellulose, resistant starch, mix-linkage glucans, hemicellulose, arabinoxylan, xyloglucan, galactomannans, pectins, non-digestible oligosaccharides, fructooligosaccharide, galactooligosaccharide, and gums.

6. The method according to claim 1, wherein the method comprises determining a dietary index using the sum of the different types of dietary fiber present in the food, meal, or diet.

7. The method according to claim 6, wherein a higher dietary index provides a more diverse gut microbiota.

8. A dietary index for assessing the effects of a food, meal, or diet on a subject's gut microbiota, wherein the dietary index is determined using the sum of the different types of dietary fiber present in the food, meal, or diet.

9. The dietary index according to claim 8, wherein the different types of dietary fiber is selected from the group consisting of: cellulose, resistant starch, mix-linkage glucans, hemicellulose, arabinoxylan, xyloglucan, galactomannans, pectins, non-digestible oligosaccharides, fructooligosaccharide, galactooligosaccharide, and gums.

10. A method for providing a dietary index according to claim 8, wherein the method comprises determining the dietary index using the sum of the different types of dietary fiber present in a food, meal, or diet.

11. A method for maintaining or improving a subject's gut microbiota diversity, wherein the method comprises:
(a) determining the effects of a food, meal, or diet on a subject's gut microbiota using a method comprising determining the sum of the different types of dietary fiber present in the food, meal, or diet; and
(b) adjusting the subject's present diet to provide an adjusted diet with an improved effect on the subject's gut microbiota diversity.

12-16. (canceled)
